(19) United States
(12) Plant Patent Application Publication
(10) Pub. No.: US 2002/0088034 P1
(43) Pub. Date: Jul. 4, 2002

(54) LILLY PLANT NAMED 'SORBONNE'

(76) Inventor: CEES A. VAN DER VOORT, KATWIJK (NL)

Correspondence Address: FOLEY & LARDNER, WASHINGTON HARBOUR, 3000 K STREET NW, SUITE 500, WASHINGTON, DC, 20007-5109, US

(21) Appl. No.: 09/357,096

(22) Filed: Jul. 19, 1999

(51) Int. Cl. A01H 5/00

(52) U.S. Cl. PLT/315

(57) ABSTRACT

A new and distinct cultivar of Lily plant named ‘Sorbonne’ characterized by having soft, pinkish-red color, white flower tips, upright flowers, and vigorous growth habit.

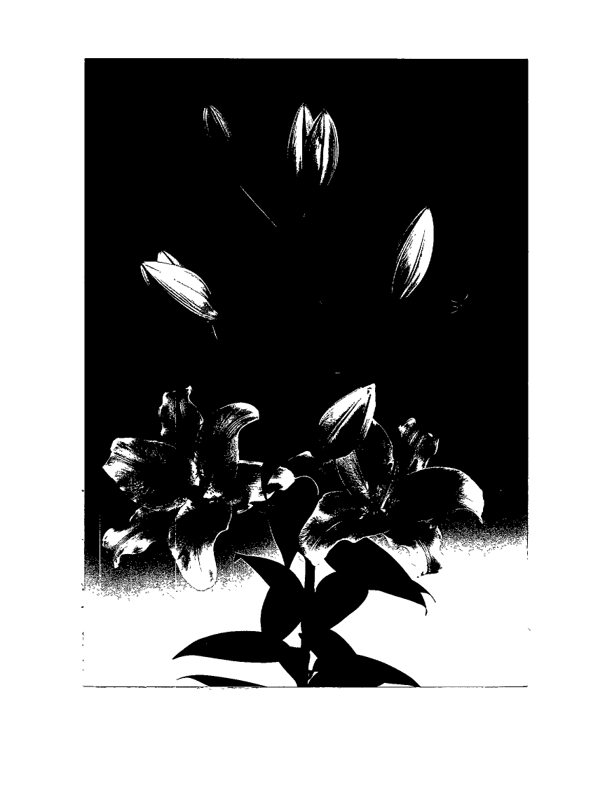

### BACKGROUND OF THE INVENTION

The present invention comprises a new and distinct cultivar of lily plant, botanically known as Lilium L., and hereinafter referred to by the cultivar name ‘Sorbonne’.

‘Sorbonne’ is a product of a planned breeding program which had the objective of creating new lily cultivars having large, upright flowers.

‘Sorbonne’ was originated from a hybridization made by the inventor in a controlled breeding program in Rijnsburg, the Netherlands in 1985. The male and female parents were unnamed seedlings of Lilium L. oriental hybrids. Both parents are proprietary cultivars used in the breeding program.

‘Sorbonne’ was discovered and selected as one flowering plant within the progeny of the stated cross by the inventor, Cees A. van der Voort, on Jun. 7, 1988 in a controlled environment in Rijnsburg, the Netherlands.

The first act of asexual reproduction of ‘Sorbonne’ was accomplished when scales were taken from the initial selection in October 1988 in a controlled environment in Rijnsburg, the Netherlands by, or under the supervision of, Cees A. van der Voort. Horticultural examination of selected units initiated in May, 1988 has demonstrated that the combination of characteristics as herein disclosed for ‘Sorbonne’ are firmly fixed and are retained through successive generations of asexual reproduction.

### BRIEF DESCRIPTION OF THE INVENTION

The following traits have been repeatedly observed and are determined to be basic characteristics of ‘Sorbonne’ which in combination distinguish this lily as a new and distinct cultivar:

1. soft, pinkish red-color;

2. white flower tips;

3 . upright flowers; and

4. vigorous growth habit.

‘Sorbonne’ has not been observed under all possible environmental conditions. The phenotype of the new cultivar may vary significantly with variations in environment such as temperature, light intensity, and daylength without any change in genotype. The following observations, measurements, and values describe the new cultivar as in the experimental research station CPRO-DLO, Wageningen, the Netherlands under conditions which approximate those generally used in commercial practice.

Of the many commercial cultivars known to the present inventor, no other cultivars are similar in comparison to ‘Sorbonne’.

### BRIEF DESCRIPTION OF THE DRAWING

The accompanying color photographic drawing shows typical plant, bulb and flower characteristics of ‘Sorbonne’, with colors being as true as possible with illustrations of this type.

### DETAILED BOTANICAL DESCRIPTION

In the following description, color references are made to The Royal Horticultural Society Colour Chart (RHS). The color values were determined between 12 am and 2 pm in July, 1993 at the Dutch experimental research station CPRO-DLO, Wageningen, the Netherlands.

| | |
|---|---|
| ORIGIN: | Seedling from breeding program |

PARENTAGE:

| | |
|---|---|
| Female parent: | Unnamed seedling |
| Male parent: | Unnamed seedling |

CLASSIFICATION:

| | |
|---|---|
| Botanical: | A hybrid of the genus Lilium L. |
| Commercial: | Oriental Lily cv. ‘Sorbonne’ |

PLANT:

| | |
|---|---|
| Form: | A single stem carrying numerous, |
| | alternately arranged pedicels |
| Height: | Medium to high for the mature plant, |
| | approximately 120 cm, depending on |
| | cultivation conditions |
| Growth Habit: | Vigorous |
| Stem: | Circular in transverse section; anthocyanin |
| | pigmentation absent; internodal length |
| | uniform |
| Foliage: | |
| Quantity: | Approximately 70 |
| Size of leaf: | Medium length and width depending on |
| | cultivation conditions |
| Shape of leaf: | Generally ovate |
| Color: | Medium green |
| Bulb: | |
| Size: | 16 to 18 cm in circumference |
| Color: | White/pink |

BUD:

| | |
|---|---|
| Form: | Tapering like a lancet |
| Size: | Medium, 11 cm in length depending on |
| | cultivation conditions |
| Opening: | Unfolds normally |
| Color: | Pinkish-green; light-pink, RHS 62 B, just |
| | before opening |
| Tepals: | Folded, three visible |

FLOWER:

| | |
|---|---|
| Blooming habit: | Raceme |
| Size: | Medium, 20 cm in diameter depending on |
| | cultivation conditions |
| Borne: | Upright to horizontal along a longitudinal |
| | axis |
| Shape: | Generally, hexagonal star in form and |
| | bowl-shaped in cross-section |
| Tepalage: | |
| Number of Tepals: | 6 |
| Arrangement: | Three inner and three outer |
| Color: | |
| Upper Surface: | Pinkish-red, RHS 64D; mid-vein is green |
| | fading to white |
| Lower Surface: | Pink, RHS 62B |
| Spotting or Marking: | Spots and markings are present, dark pink |
| | in color |
| Pedicel: | |
| Length: | Medium, 10 cm long |
| Color: | Soft pink, RHS 65A, at the tips of the |
| | tepals fading to white |
| Form: | Tapering like a lancet |
| Texture: | Papillose |
| Disease Resistance: | Medium |
| Fragrance: | Weak |
| Lasting Quality: | Excellent |

REPRODUCTIVE ORGANS:

| | |
|---|---|
| Stamens and anthers: | |
| Arrangements: | Anthers form a generally circular pattern |
| | at their upper ends |
| Number: | 6 |
| Length: | Medium length depending on cultivation |
| | conditions |
| Filaments Color: | Green |
| Pollen color: | Orange-brown |
| Anther color: | Orange-brown |
| Pistils: | Green |
| Stigma: | Purple |
| Ovaries: | Triangular in shape |

I claim:

1. A new and distinct cultivar of Lily plant named ‘Sorbonne’, as illustrated and described herein.
